(19) United States
(12) Patent Application Publication
(10) Pub. No.: US 2020/0410023 A1
(43) Pub. Date: Dec. 31, 2020

(54) SYSTEM AND METHOD OF IDENTIFYING, AND MAKING AVAILABLE, INFORMATION RELATING TO PERSONS OR EVENTS

(71) Applicant: Project Legacy Pty. Ltd., Kent Town (AU)

(72) Inventor: James Edward HURST, North Adelaide (AU)

(21) Appl. No.: 16/947,011

(22) Filed: Jul. 15, 2020

Related U.S. Application Data
(63) Continuation-in-part of application No. 15/761,039, filed on Mar. 16, 2018, now abandoned.
Application No. 15/761,039 is a continuation-in-part of application No. PCT/AU2019/050108, filed on Feb. 12, 2019.

(30) Foreign Application Priority Data
Sep. 17, 2015 (AU) 2015903802
Mar. 15, 2016 (AU) 2016900969
Feb. 12, 2018 (AU) 2018900426
Feb. 13, 2018 (AU) 2018900451

(51) Int. Cl. G06F 16/9535; G06F 16/29; G06F 16/587; G06F 16/23; G16H 10/60; G06F 16/9538

(52) U.S. Cl. CPC G06F 16/9535; G06F 16/29; G06F 16/9538; G06F 16/2379; G16H 10/60; G06F 16/587

(57) ABSTRACT

A computer implemented system enabling information relating to persons or events to be made available by a user to one or more other users, the system including a user interface, a coordinate system, and a mapping application, wherein the user interface enables the user to navigate the coordinate system by the use of the mapping application to identify the person or event, and enter, amend and/or delete information in a database, relating to the person or event.

### FIELD

The present invention relates to a system and method for enabling information relating to persons or events, such as, but not limited to, deceased family members or significant events in history, to be made available, to, for example, the general public.

### BACKGROUND

Computer software applications providing systems and methods for searching and identifying information regarding persons of interest (for example, family ancestors) or events in history are presently available. One example is ancestry.com, which is an online application that enables a user to locate and gather information regarding their genealogy. Such information is extracted from existing and publicly accessible records and/or from official registries such as the Registry of Births, Deaths and Marriages.

However there are many countries in which accurate records have not been maintained or have been lost due to, for example, war, natural or man-made disasters, social and economic oppression. As a result, it is often difficult for many people to trace their genealogical history or find lost family members using the internet or applications such as ancestry.com, since official records, such as Birth, Marriage and Death Certificates, either do not exist, have not been maintained or have not been accurately recorded.

In addition, the personal records maintained in presently available computer software applications are not able to be updated by persons having direct knowledge and/or experience regarding family members, and hence, the records only reflect official records and information that is publicly available such as published historical information. Accordingly, information regarding family ancestors is often not recorded, and as a consequence, much of our history is being lost.

Another challenge that is often encountered by persons seeking to learn about their genealogy, is a lack of knowledge with respect to names of present and/or deceased family members or family associates. Accordingly, in order to successfully identify information regarding a family member or ancestor, a searcher is required to have accurate details including a name of the person they seek to identify. This presents difficulties in identifying and/or locating people in situations where people change their names, either through marriage or other circumstances, or when people have common names. Such difficulties make identifying/locating a person problematic and even sometimes impossible.

In addition, conventional methods and systems for identifying information relating to persons or events of interest also make searching for people and events difficult, if not impossible, due to the vast volume of information captured and available on data communication networks such as the internet. Searching with the use of applications such as Google, often returns many results that are not relevant to the person of particular interest to the searcher.

A further disadvantage associated with conventional methods and computer software applications for identifying information relating to persons and events of interest is that the searcher is often required to filter a vast amount of information available on the internet which requires the searcher to enter specific search criteria in searching tools which often yields incorrect results due to the entry of inappropriate search criteria. Accordingly it is difficult, if not impossible, to identify information regarding persons or events of interest.

A still further disadvantage is that the majority of information publicly available with regard to a particular event is generated by the media, and hence is often not an accurate representation of the facts associated with the event. Presently, there are limited opportunities in which any member of the public can provide their personal account and/or experience of an event, and if they do record it, for example, on the internet, the same issues associated with identifying such information exists for reasons discussed above.

Accordingly, difficulties presently exist associated with the ability to identify, locate and contact persons within an associated extended network or group of people. Difficulties also presently exist regarding the ability to add information to a body of knowledge, associated with persons or events of interest.

The present invention is intended to address at least some of the above disadvantages associated with conventional methods of identifying and recording information associated with persons or events of interest.

The reference to any prior art in this specification is not, and should not be taken as, an acknowledgement or any suggestion that the prior art forms part of the common general knowledge.

### SUMMARY

In one aspect, the present invention provides a computer implemented system enabling the discovery of personal contact details relating to a first user associated with a person or event of interest, the system including, a user interface, a geographical image, and a mapping application, wherein the user interface enables the first user to, enter their personal contact details to the geographical image for discovery by one or more second users, navigate the coordinate system by the use of the mapping application to identify a person or an event of interest, the first user able to identify the person or event of interest without requiring, prior to navigating the coordinate system, the name of the person or event, and enter, amend and/or delete information in a database, relating to the identified person or event including a date or date range associated with the information, and wherein the user interface enables the one or more second users to, enter a second date or date range of interest into a search field and navigate the geographical image using the mapping application according to the second date or date range, and thereby discover the entered personal details pertaining to the first user when the second date or date range matches the first date or date range.

In another aspect, the present invention provides a computer-implemented method for enabling the discovery of personal contact details relating to a first person associated with a person or event of interest, the method including, the first user, accessing, by a user interface configured to display a geographical image, a computer-implemented system, navigating the coordinate system, by the use of a mapping application, to identify a person or an event of interest, the user able to identify the person or event of interest without requiring, prior to navigating the coordinate system, the name of the person or event, and entering, amending and/or deleting information relating to the identified person or event in a database using the mapping application associated with the coordinate system, and the one or more second users, entering a second date or date range of interest into a search field and navigating the geographical image using the mapping application according to the second date or date range, and thereby discovering the entered personal details pertaining to the first user when the second date or date range matches the first date or date range.

In another aspect, the present invention provides a computer implemented system enabling information relating to persons or events of interest to be entered, amended and/or deleted by one or more users, the system including, a user interface, a coordinate system, and a mapping application, wherein the user interface enables a user of the one or more users to, navigate the coordinate system by the use of the mapping application to identify a person or an event of interest, the user able to identify the person or event of interest without requiring, prior to navigating the coordinate system, the name of the person or event, and enter, amend and/or delete information in a database, relating to the identified person or event.

In yet another aspect, the present invention provides a computer implemented system enabling the discovery of personal contact information pertaining to a first user, the system including, a user interface for use by first and second users, a geographical image, a mapping application, wherein the user interface enables a first user to anchor their personal contact information and a first date or date range to the geographical image for discovery by one or more second users, and wherein the user interface enables the one or more second users to enter a second date or date range of interest into a search field to search the geographical image using the mapping application thereby discovering anchored personal contact information pertaining to the first user when the second date or date range matches the first date or date range.

### Additional Aspects of the Invention

### System of the Invention

In another aspect, the present invention provides a computer implemented search system for information pertaining to a first user, the system including: a first user interface for use by first users; an image; and a mapping application; wherein the first user interface enables a first user to anchor information to the image for discovery by one or more second users; and wherein a second user interface enables one or more second users to search the image by use of the mapping application to discover anchored information pertaining to one or more first users.

In another aspect, the present invention provides a computer implemented search system for persons of interest, the system including a first user interface, a coordinate system, one or more oceans of information, and a mapping application, wherein the first user interface enables one or more first users electing to be discoverable to anchor (pin or otherwise enter) information to the one or more oceans, and wherein a second user interface enables one or more second users to trawl (or search) one or more oceans of information associated with the coordinate system by use of the mapping application to discover information pertaining to one or more first users anchored to the one or more oceans.

In another aspect, the present invention provides a computer implemented search system for objects associated with persons of interest by a user, the system including: a first user interface; an image associated with one or more objects; and a mapping application; wherein the first user interface enables a first user electing to be discoverable to anchor information to the image; and wherein a second user interface enables a second user to search the image by use of the mapping application to discover information pertaining to the first user anchored to the image.

In another aspect, the present invention provides a computer implemented search for objects associated with persons of interest by a user, the system including a first user interface, an image of one or more objects, and a mapping application, wherein the first user interface enables a first user electing their object to be discoverable to anchor information to an object in the image, and wherein a second user interface enables a second user to search the image by use of the mapping application to discover information pertaining to the first user anchored to one or more objects in the image.

In another aspect, the present invention provides a computer implemented searching method for information pertaining to a first user by one or more second users, the method including: the first user accessing a computer-implemented system through a first user interface, wherein the system includes an image and a mapping application; the first user navigating the image using the mapping application associated with the image; and the first user anchoring information to the image that enables the discovery of information pertaining to the first user by the one or more second users.

In another aspect, the present invention provides a computer-implemented searching method for discovering a first user by one or more second users, the method including the steps of: the first user accessing a computer-implemented system through a first user interface, wherein the system includes a coordinate system, one or more oceans of information and a mapping application; and the first user navigating the coordinate system using the mapping application associated with the coordinate system; and the first user anchoring information to one or more oceans of information using the mapping application associated with the coordinate system, wherein the information enables the discovery of the first user by the one or more second users.

In another aspect, the present invention provides a computer-implemented searching method discovering objects associated with a first user, the method including the steps of: a first user accessing a computer-implemented system through a user interface, wherein the system includes an image associated with one or more objects and a mapping application; and the first user navigating the image using the mapping application; and the first user anchoring information to the image using the mapping application, wherein the information enables the discovery of objects associated with the first user by the one or more second users.

In another aspect, the present invention provides a computer implemented searching method enabling one or more second users to discover information pertaining to a first user, the method including: the one or more second users accessing a computer-implemented system through a user interface, wherein the system includes an image and a mapping application; and the one or more second users searching for information using the mapping application to locate and view one or more virtual buoys of information anchored to the image.

In another aspect, the present invention provides a computer implemented searching method enabling one or more second users to discover a first user, the method including the steps of: the one or more second users accessing a computer-implemented system through a user interface, wherein the system includes a coordinate system, one or more oceans of information and a mapping application; and the one or more second users trawling for information using the mapping application associated with the coordinate system to locate and view one or more virtual buoys of information anchored within the one or more oceans.

In another aspect, the present invention provides a method enabling one or more second users to discover one or more objects associated with a first user, the method including: the one or more second users accessing a computer-implemented system through a user interface, wherein the system includes an image associated with the one more objects and a mapping application; and the one or more second users searching the image using the mapping application to locate and view one or more virtual buoys of information anchored to the image.

In another aspect, the present invention provides a computer implemented system enabling information relating to persons or events to be made available by a user to one or more other users, the system including: a user interface, a coordinate system, and a mapping application, wherein the user interface enables the user to, navigate the coordinate system by the use of the mapping application to identify the person or event, and enter, amend and/or delete information in a database, relating to the person or event.

In another aspect, the present invention provides a computer implemented system enabling information relating to persons or events to be identified by one or more users, the system including: a user interface; a coordinate system; and a mapping application; wherein the user interface enables the user to: navigate the coordinate system by the use of the mapping application to identify information relating to the person or event; wherein the information relating to the person or event includes a network of associations associated with the person or event.

In another aspect, the present invention provides a method for enabling information relating to persons or events to be made available by a user to one or more other users, the method including: the user accessing a computer-implemented system through a user interface, wherein the system includes a coordinate system, and a mapping application, the user navigating the coordinate system using the mapping application associated with the coordinate system to identify the person or event, and the user entering, amending and/or deleting information into a database relating to the person or event using the mapping application associated with the coordinate system.

In yet another aspect, the present invention provides a method for enabling information relating to persons or events to be identified by one or more users, the method including: the user accessing a computer-implemented system through a user interface, wherein the system includes a coordinate system, and a mapping application, the user navigating the coordinate system using the mapping application associated with the coordinate system to identify the person or event, wherein the information relating to the person or event includes a network of associations associated with the person or event.

### Embodiments of the Invention

In an embodiment, a first user desiring to be discovered, anchors their information to the one or more oceans by the use of a virtual buoy.

In an embodiment, each virtual buoy has one or more attributes that allow filters, or search criteria, to be applied or entered by a second user during the trawling process. Such attributes may include, but are not limited to, dates, geographic locations and events or places of interest.

Accordingly, when a second user is trawling for information, the second user may select a particular ocean to trawl and may enter one or more search criteria to be applied to the various attributes.

In an embodiment, the one or more oceans of information may be made visible by a first user, or may be hidden by a first user, from one or more second users accessing the system.

In an embodiment, the mapping application allows a user to trawl one or more oceans of information in which the user is able to use an electronic input device and cursor to cast, or drag, a virtual net over a defined geographical area to thereby limit search results to only the selected area.

In an embodiment, a first user desiring an object to be discovered may anchor their information to an object in the image by the use of a virtual buoy.

In an embodiment, each virtual buoy has one or more attributes that allow filters, or search criteria, to be applied or entered by a second user during the searching process. Such attributes may include, but are not limited to, dates, geographic locations and travel distances to the location of the object. Accordingly, when a second user is searching for information, the second user may select a particular image to search and enter one or more search criteria to be applied to the various attributes.

In an embodiment, the mapping application allows the second user to use an electronic input device to position a cursor over a specific location on the image and view information anchored to that specific location by a first user.

In an embodiment, the mapping application allows a user to search by “trawling” in which the user is able to use their cursor and an electronic input device to cast a virtual net over an area of the image in order to limit search results to only the selected area.

The system according to aspects of the invention may be a publicly accessible system. Alternatively, the system of the invention may be a private system requiring authentication of first and/or second users to access and use the system.

The system may be computer-implemented system which users may access via a data communications network, such as the internet, or the system may be an application associated with a particular device.

The system may be a separate or stand-alone application that provides all of the functional features or elements of the system, or alternatively, the system may be embedded within one or more external (third party) systems or may itself offer additional functionality to an external (third party) application. Typical third party applications that are envisaged as being particularly suited for use in the system of the present invention are mapping applications such as Google or Bing maps, and social/professional networking sites such as Facebook or LinkedIn.

The system of the invention according to any embodiment may be a single application or alternatively, may be a distributed application wherein separate applications are executed on separate computer processors to implement one or more features separately.

The user interface may be any conventional interface system that operates on a number of electronic devices including, but not limited to, a personal computer, laptop, mobile phone or iPad, and one that allows a user to interact with and use the system of the invention. The user interface may also allow authentication of a user's details by requiring the user to enter, for example, a unique username and password in order to access the system.

Authentication may be required in situations in which a user seeks to update information, hide information, make information visible to one or more other users and/or anchor information to the coordinate system. Alternatively, authentication may be required when users seek to access private “oceans” of information with restricted access. Of course, authentication may not always be required, particularly in situations where a user seeks to search (or “trawl”) oceans of information that are visible and publicly accessible.

### Discovery Through Anchoring and Trawling

Information may be anchored by a first user seeking to make themselves, or an object in their possession, “discoverable” by means of one or more virtual buoys which are discrete information records associated with the first user. Each buoy may contain the anchoring position of the information on the coordinate system and provide the spatial context for the information and associate the data record with a location. For example, a person that attended a particular college at a certain point in time may anchor their information to the geographical location of that college thereby making themselves “discoverable” to other users of the system.

The information anchored by the first user making themselves, or an object in their possession, available to be discovered may include details such as their name, their contact details such as a telephone number, mobile phone number or current address. The contact details may also be an email address or a Skype address or may be a link (for example a URL, path or pointer) to the first user's profile on a social or professional networking site such as Facebook or LinkedIn. Ultimately, the contact details, or link, provide sufficient information to facilitate contact between two parties (i.e., the first and second users).

In an embodiment, the security features associated with a third party social networking website such as Facebook or LinkedIn will provide any required level of security and privacy to users.

Additionally, each virtual buoy (or information record) may include one or more attributes, such as, but not limited to, names and dates, that assist in the trawling (or searching) process. It will be appreciated that the one or more attributes should make searching easier since the user is able to focus the trawl (or search) and thereby limit the amount of information that is discovered as a result of the trawl (or search).

In an embodiment, the attributes associated with each virtual information buoy are determined by the ocean in which the virtual buoy is anchored. For example, an ocean containing information regarding people would have different attributes as compared with an ocean containing information in respect of places of interest. In addition to names and dates, other possible attributes that assist trawling for information associated with persons of interest may include keywords, geographic location or details pertaining to an event.

Typically, one or more virtual information buoys will be anchored in a defined ocean of information, wherein each virtual buoy is linked to records of a person of interest and is therefore “discoverable” to a user trawling (or searching) for information.

Desirably, the system of the invention is able to be used by persons of limited computer skills such that a second user is able to easily “trawl” for information or a first user is able to simply drop an “anchor” of information at any point on a coordinate system.

### The Geographic Coordinate System and Mapping Application

In an embodiment, the coordinate system is a geographic coordinate system and provides a reference upon which “virtual buoys” may be anchored within one or more defined oceans. The geographic coordinate system may have one, two or multiple dimensions depending upon the application. A typical example of a possible geographic coordinate system is the global spherical (latitudinal/longitudinal) coordinate system used to specify the position of a point on the earth's surface. Other geographic coordinate systems include map projection models that are used to project maps of the earth's spherical surface onto a two-dimensional Cartesian (X/Y) coordinate plane.

A mapping application may be associated with the image or the geographic coordinate system that may overlay an image, a geographic map or other visual or audio content associated with the geographic coordinate system thereby allowing a second user to navigate the geographic coordinate system to trawl or a first user anchor information with respect to one or more oceans of information.

Information may be anchored by a first user onto one or more oceans associated with a geographic coordinate system using a mapping application such as, but not limited to, Google Maps, Apple maps or Bing maps.

The mapping application may also provide functionality that enables a second user to trawl (or search by moving their electronic input device and cursor to thereby drag a virtual net over a defined geographic area) and locate a point of interest on the geographic coordinate system at which point information may have been anchored by a first user. In its broadest form, mapping applications may also include applications and devices such as media players, image viewers and televisions.

### The One or More Oceans of Information

The term “ocean” in the context of the present specification means the logical or physical separation of data into any desired categories. As an example, such categories may include, but are not limited to, information relating to certain individuals or different groups, or information relating to certain events associated with a particular date range. Of course, the number of oceans and their purpose will depend upon the nature of the information through which a first and second user seek to contact each other.

It will be appreciated that the physical separation of data into one or more oceans assists maintaining a logical separation of data that enables a single system to be used for a variety of purposes and also allows the one system to cater for different user groups and also assists to avoid overcrowding of data.

An “ocean” of information may be publicly accessible or may be private and therefore accessible to only a select group of users who have been provided authorized access to that particular ocean of information. In this regard, various groups may be able to offer information to a select group of users or a single user may be able to make themselves discoverable to only a select group of users. This is preferably achieved by enabling a first user who anchors their information to “turn on” (make visible) or “turn off” (hide) one or more oceans completely or partially. For example, a first user may prefer to make their contact details visible to only a select group of people and so may make the ocean partially visible to a select group of second users.

It will be appreciated that embodiments of the invention may use one or more oceans which a second user may be able to “turn on” or “turn off” as required in order to access and view different layers of information.

### Representation of an Image and Mapping Application

In an embodiment, images include, but are not limited to, objects such as an automobile, a furniture item, a house, or a computer.

The image may be any type of image and includes, but is not limited to, a graphical image, a wire-frame image, a 3D virtual model or a photograph.

In an embodiment, the image is overlaid with a coordinate system that enables the user, through the use of the mapping application associated with the image, to identify and/or select an object, or part of an object, located in the image. In an embodiment, the coordinate system is a grid coordinate system and provides a reference upon which “virtual buoys” may be anchored within the image. The grid coordinate system may have one, two or multiple dimensions depending on the application.

### User Anchoring of Information

In embodiments where more than one ocean of data exists, the first user may also select one or more oceans of interest within which to navigate and anchor one or more virtual buoys.

In an embodiment, the first user may invoke a function to drop an anchor at a position of interest. This may involve clicking, tapping or invoking a menu function with an electronic input device. The first user may also populate fields with attributes that will be associated with a virtual buoy of information that will allow the user to be discovered according to search criteria. It will be appreciated that in certain embodiments, the computer system may be able to automatically populate various fields with attributes, for example, the first user's name associated with their log-in details.

The first user may also provide a link or some pointer to a third party website such as their social or professional networking profile such as Facebook or LinkedIn.

### User Trawling to Discover a Person of Interest

In embodiments where more than one ocean of data exists, the second user may also select one or more oceans of interest within which to conduct a trawl (or search).

In situations where large amounts of information exist, a second user may also enter one or more search criteria restricting their search to attributes associated with a first user to limit the trawl area. For example, a second user may limit the trawl area by keyword, geographic location and/or date range.

Upon completing a trawl, the second user may be presented with one or more results (virtual buoys) that match the entered search criteria. At this point, the second user may be able to inspect the virtual buoys and identify contact information if attributes such as a name and a telephone number are associated with the virtual buoy. Alternatively, if a virtual buoy that matches the trawl criteria contains a link to a social website such as Facebook, the second user may select or otherwise use the link to be transferred to the website of the discovered first user.

### System and Method Enabling Users to Update Information in Relation to Other Persons or Events

In an embodiment, the information relating to the person or event includes a genealogical chart (sometimes known as a family tree) associated with the person or event.

In an embodiment, the genealogical chart displays the contact details of one or more members within the genealogical chart. In this way, contact and communication between family members, who may or may not be known to each other, can be initiated/facilitated as a result of sharing a common ancestor.

In an embodiment, the information relating to a person includes a medical history of the person. The medical history may also include details regarding a family medical history and may further include details regarding the family history pertaining to one or more members within a genealogical chart. Accordingly, users (i.e., family members) are able to access medical information with respect members of their extended family, whether such persons are known to them or not. It will be appreciated that the ability to access a medical history regarding family members (including family members that are not known to the user) may be particularly useful for persons seeking to gain an understanding regarding their own personal health and any potential medical risk factors or characteristics. In this regard, genetic diseases such as heart disease, breast/bowel cancer and other various auto-immune diseases are often passed onto descendants. It will also be appreciated that the ability to access a medical history regarding family members (including unknown and deceased family members) may also result in improved or expedited diagnosis of disease and may also prompt a user to seek medical attention/intervention earlier than they may otherwise. This is particularly useful for any medical conditions where early diagnosis dramatically improves patient outcomes, for example, breast cancer.

In another embodiment, the medical information includes general health (such as height and weight from birth to adulthood), behavioral traits and characteristics which may be of particular interest to distant family members.

In an embodiment, the information relating to the person includes a cause of death. This may be in the form of, for example, text or a death certificate. The information relating to the cause of death may pertain to one or more members within a genealogical chart. Accordingly, in this embodiment, users (i.e., family members) are able to access information relating to the cause of death with respect to members of their extended family, whether such persons are known to them or not.

In an embodiment, the user interface also enables a user who enters information regarding a person or event, to also enter their own contact details and therefore make themselves contactable by one or more other users.

The contact details may include, but are not limited to, any one or more of a name, telephone number, email address, postal address, or a link to professional or social networking site such as a LinkedIn profile or Facebook page.

The user interface, in an embodiment, may also enable one or more users to search the coordinate system by the use of the mapping application in order to search for, and identify, information pertaining to a person or event that has been entered by another user.

For example, if a user desires to locate information relating to their genealogy, yet only has limited information, e.g., a geographical region in which their surname is popular (for example, the surname “Rossini” is popular in the north of Italy), the user can navigate to that known geographical location using the mapping application of the present invention, and identify any information associated with persons having the same surname and similar profiles/family trees. It will be understood that such information may have been entered or “pinned” to a particular location by one or more other users according to the system and method of the present invention.

It will also be appreciated that such information can include, but is not limited to, family trees, historical accounts relating to various family members and/or family events, photographs, video material, eulogies, obituaries, biographies, autobiographies, and contact details of various family members including links to various social or professional networking sites, for example, Facebook and LinkedIn.

In an embodiment, the coordinate system is associated with a geographical map.

In an embodiment, the one or more users are able to enter information, and/or search the coordinate system, according to a location, date, or date range.

For example, a user can enter or “pin” information at a particular geographic location and at a particular point in time (or time period) with regard to a deceased family member. The user can enter details such as the place and date of death of the family member and write or attach, for example, their family tree, a eulogy about that family member and their personal contact details in the event they desire to be contacted by one or more other users. It will be appreciated that this information will remain as a historical record, that can be accessed and viewed by anybody having an interest in the user, or the user's deceased family member.

It will also be appreciated that one or more users of the system and method of the invention may also able to enter, amend and/or delete information relating to a person or event in respect of which information has been entered by another user.

Accordingly, if another user navigates, in this embodiment, a geographical map using the mapping application of the present invention and identifies information regarding a person of interest that happens to be the deceased family member discussed in the example above, if the other user also knew that family member and preferred to add to the body of knowledge and accessible information regarding the deceased person, the user could either contact the user who initially posted information regarding their deceased family member, and request permission to update the deceased persons profile. It will be appreciated that, in this embodiment, permission is required to be sought by another user from the user who initially pinned in the information in order to update or amend such information. It will be appreciated, however, that in other embodiments, the present invention contemplates the updating of information located in the database by a user and in the absence of any permission.

For example, if a user desired to provide and share an account of their own personal experience regarding a significant event in history (for example, the US (“9/11”) terror attack in New York on the 11th of September 2001) the user could navigate to the geographic location: 285 Fulton Street, New York, N.Y. 10007, USA, select the date 11 Sep. 2001, and post their comments, photographs and contact details.

Accordingly, the present invention in various embodiments assists in capturing historical information associated with persons and events, present or historical, according to the knowledge and/or experience of members of the public associated with such persons or events. It will be appreciated that this is expected to provide a more accurate and intimate account of persons and events, as opposed, to information published by groups such as, for example, the media or government bodies.

It will be appreciated that the network of associations may include any type of group or association such as, for example, a sporting group or members of a graduating class that attended a particular educational institution.

In an embodiment, the network of associations may also include the contact details of one or members within the network. The contact details may include, but are not limited to, any one or more of a name, telephone number, email address, postal address, or a link to a professional or social networking site such as a LinkedIn profile or Facebook page.

In an embodiment, the network of associations may include a dendritic association, for example, a genealogical chart in which the branched relationship between various members of a family are shown.

In an embodiment, once information relating to a person or event has been identified by the one or more users, the one or more users are also able to enter, amend and/or delete information in a database relating to the person or event.

### BRIEF DESCRIPTION OF THE DRAWINGS

One or more embodiments of the present invention will now be described with reference to the accompanying figures in which:

FIG. 1 provides a conceptual illustration of a system in accordance with an embodiment of the invention identifying primary system components and how the components relate to one another to allow a user to either enter, or search for, information relating to a person or event;

FIG. 2 provides a conceptual illustration detailing the association between the mapping application and a coordinate system and also illustrating the entry of a user's personal information into a database in accordance with an embodiment of the invention;

FIG. 3 illustrates a list of possible attributes that may be associated with an information “pin” with a description/purpose of each attribute in addition to an example of each possible attribute;

FIG. 4 provides a conceptual illustration depicting how attributes are linked to an information pin to provide more information associated with a person, or an event, to facilitate interaction between two parties;

FIG. 5 provides an example of a user interface in accordance with an embodiment of the invention detailing an embedded mapping application, various “pinned” information and the contact details of a person who pinned the information and the search filter adopted by a user to limit and/or focus a search;

FIG. 6 is a block diagram depicting how a system may be used in accordance with an embodiment of the invention enabling a user to “drop a pin” in order to enter information about a person, or event, in order to make such information available to one or more users;

FIG. 7 is a block diagram depicting how a system may be used in accordance with an embodiment of the invention by a user to search for a person, or event, according to a known location associated with the person or event;

FIG. 8 provides an example of a user interface that a user encounters upon attempting to access a system in accordance with an embodiment of the invention; and

FIG. 9 provides an example of a user interface showing the results of a search in accordance with an embodiment of the invention.

### DETAILED DESCRIPTION OF THE PRESENT EMBODIMENTS

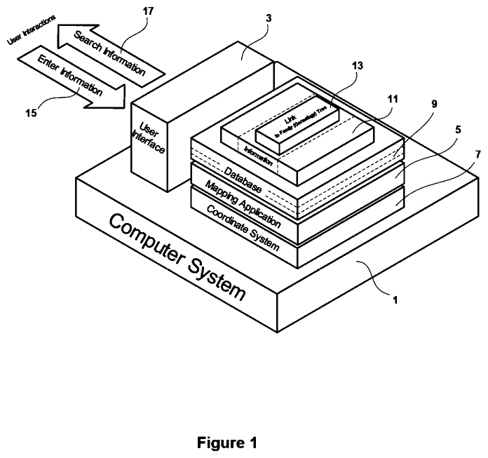

A system according to an embodiment of the invention is illustrated in FIG. 1 wherein the system components and their interrelationship is illustrated. In this embodiment, a computer-implemented system (1) that may be any device including, but not limited to, a personal computer, laptop, mobile phone or iPad, is accessed through user interface (3) and includes mapping application (5) associated with geographic coordinate (grid/coordinate) system (7), Mapping application (5), for example Google Maps, allows a user to search one or more databases (9) of information in order locate one or more information pins (11) associated with a person or event of interest. FIG. 1 also shows that one or more buoys (11) is/are associated with one or more links (13) to any third party website including professional or social networking sites such as, but not limited to, Facebook and/or LinkedIn.

The illustration of FIG. 1 also depicts an inwardly facing arrow (15) that demonstrates the entry of information by a user onto system (1) and in particular in database (9) in relation to a person (which could be themselves) or an event. FIG. 1 also depicts an outwardly facing arrow (17) that demonstrates searching by another user when the other user seeks to identify any information made available in relation to a person or an event of interest.

Any first time user seeking to enter discoverable personal information must initially register on the system (1) and create a profile by providing, as a minimum, a user name and password. In order to re-enter the system (1), the user will necessarily undergo an authentication process and re-enter their username and password thereby ensuring a secure system that only allows a user's personal details and/or discoverable information to be updated/altered after entry of the correct authentication details. Of course, if a user only seeks to search for information regarding persons of interest, it may not be necessary to create a profile and the user may access the system (1) in the absence of any authentication step.

The system (1) shown in FIG. 1 may be a computer system, one or more computer applications, or any device with a user interface that allows a user to access the system (1). The system (1) may be a stand-alone system that provides all the necessary functionality to allow a user to access, search and enter (or “pin”) information. In other embodiments, the system (1) may be embedded within another third party system, such as Google Maps, that provides either all, or at least some, of the features/applications to allow a user to search and pin information. Alternatively, the system (1) may provide additional functionality to an existing third party application such that it enables a user to search and pin information. The system (1) may be publicly accessible through the internet, or it may be a private program with restricted access.

It will be appreciated that the system and method of the present invention addresses the difficulty that confronts many people when attempting to identify information regarding family members, alive and deceased, using data communication networks such as the internet, conventional search tools such as the telephone directory, or specific genealogy subscription-based websites such as ancestry.com. Searching for information regarding persons or events of interest is often tedious and can often yield either no results or a vast amount of results that, due to the large volume, ceases to be useful or provide any assistance with respect to locating such information.

In an embodiment, the system and method of the invention enables information relating to persons or events of interest to be searched geographically. Accordingly, any user seeking to identify information relating to one or more persons or events of interest, can conduct a search on the basis of a geographic area or location that is known to have an association with the person or event of interest.

The following description relates to a specific embodiment of the system and method of the invention. However, it will be appreciated by skilled readers that the invention is not limited to the specific features of the described system and method.

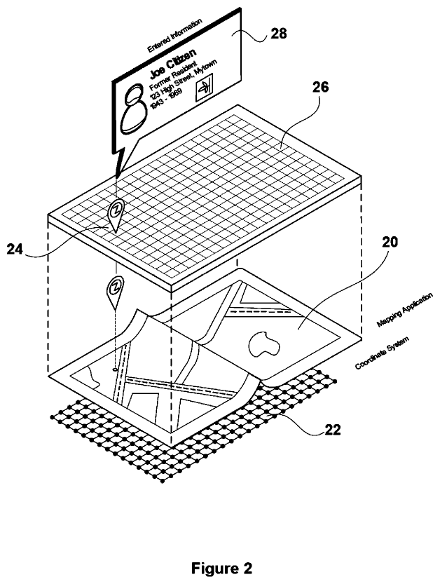

In the embodiment shown in FIG. 2, the system is constructed as a stand-alone computer system that embeds a mapping application (20) such as Google maps. Google maps is particularly suited for use in the described embodiment of system of the present invention as it will be appreciated that this application operates on a variety of platforms and has an extensive application programming interface (API), wherein the API enables Google maps to be embedded within other applications and also allows other features/applications to be overlaid on the maps. Whilst Google Maps is a preferred mapping application, other mapping applications such as Apple or Bing maps may be used with the system and method of the present invention.

The mapping application (20) provides the functionality that allows a user to identify information relating to a person or event of interest on a map (geographic coordinate system (22)) where one or more information pins (24) are operable to be “dropped” into one or more databases (26) of information (28). Additionally, the mapping application (20) also allows a user to locate the area upon which they intend to search for any information relating to pins that have been “dropped” by other users.

In the embodiment shown in FIG. 2, the geographic coordinate system (22) uses the global spherical (latitudinal/longitudinal) coordinate system used to specify the position of a point on the earth's surface. It will be appreciated that other coordinate systems may be suitable for use in the system of the present invention including map projection models that are typically used to project maps of the earth's spherical surface onto a two-dimensional Cartesian (X/Y) coordinate plane. Other coordinate systems that may be used within the system of the present invention may have one, two or multiple dimensions depending upon the application.

In the instance that information relating to a number of persons or events occurring at different elevations with respect to a particular geographical location (e.g. different floors of a multi-story building or apartment complex), the co-ordinate system may accommodate this eventuality. Alternatively, when seeking to enter information regarding a person or event at a geographical location with more than one possible elevation, the system may prompt the user to include additional information to define the elevation pertaining to the person or event (e.g. floor number or level).

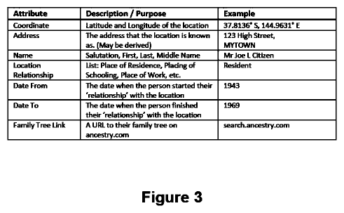

With further reference to FIG. 2, information pin (24) represents information relating to a person of interest (Joe Citizen) having an association with the location at which pin (24) was dropped by another user. An example of the information associated with a particular pin is shown in FIG. 3, in which each information pin or record within database (26) has attributes such as those shown in FIG. 3. In an embodiment, each dropped pin is implemented as a row, or record, in a spatial database.

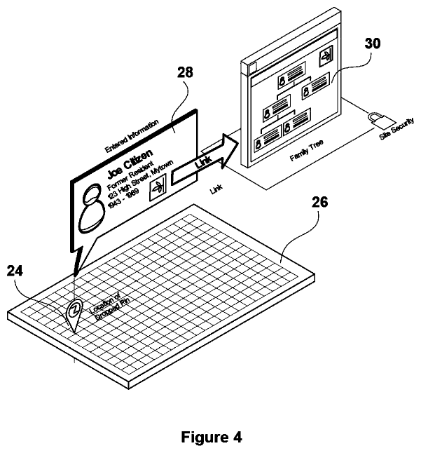

In the embodiment shown in FIG. 2, associated with the information (28) is a link (30) as shown in FIG. 4. Link (30) is implemented as a URL hyperlink to a genealogy website such as ancestry.com and is one of the attributes captured in the information pin (24) as is identified in FIG. 3. Alternatively, a user may upload a family tree directly into database (26) which may be accessed and viewed by one or more other users having an interest in the same person or event. In one embodiment, the link is implemented as a URL hyperlink to a database of personal accounts regarding an event.

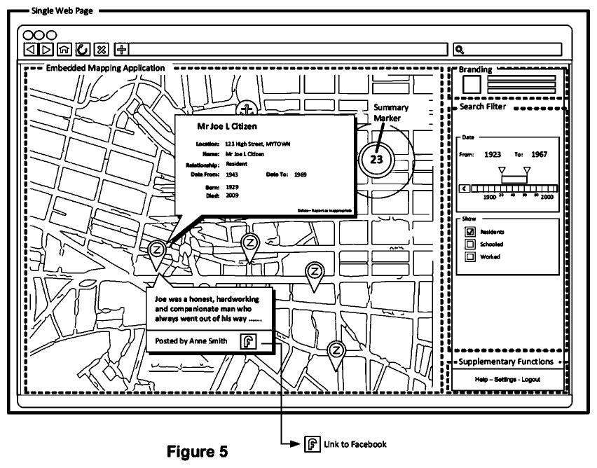

The system and method of the present invention preferably enables a user to search for, and/or enter/amend/delete, information relating to a person or event of interest in the absence of any special skills. In an embodiment, a single web page user interface enables a user to search and/or enter information regarding persons or events of interest on the same webpage. An example of an interface that may be used with the system and method of the present invention is shown in FIG. 5. It will be appreciated that the interface shown in FIG. 5 represents only one possible configuration and other interface configurations are possible.

Preferably, the user interface will support at least two user interactions including “pinning” and “searching”. In other embodiments, the user interface supports a range of additional functions including, but not limited to:
Setting user preferences/user details;
Obtaining/providing help and information;
Entering user registration details;
Logout/exit functions;
Allowing a user to update or delete pins;
Allow a user to report pins that are considered inappropriate/inaccurate; and/or
Allow a user to obtain/set a new password.

The system will also desirably have one or more security features to prevent unwanted pinning and saving of information pins by automated programs (i.e. non-human users).

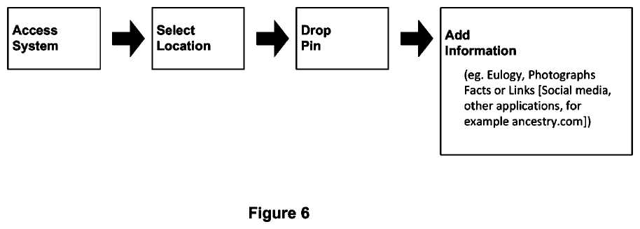

The steps involved in a user pinning information onto a system in accordance with an embodiment of the invention is shown in the block diagram of FIG. 6.

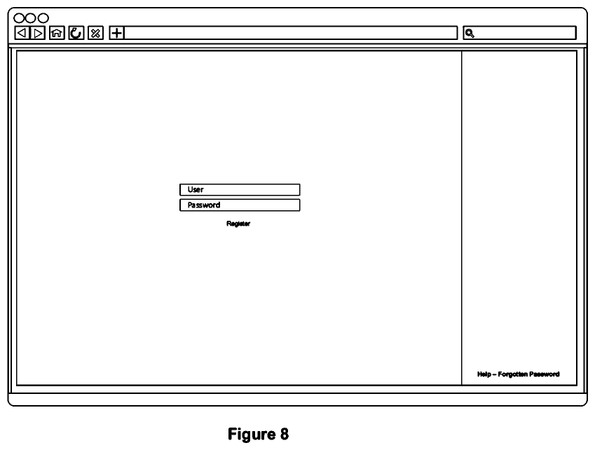

The first step involves accessing the system through a user interface associated with the system that is available across the internet. The user is able to access the system by launching a browser and entering a URL upon which the user is presented with a login page as shown in FIG. 8.

Authentication of the user is required and accordingly the user is prompted to enter their username and password. Upon providing the correct username and password details, the user is directed to a system profile which may contain personal details of the user such as their name and any links to associated social media profiles which could be used to auto-populate information pins.

Once the user is directed to their system profile, the user will typically select a geographic location in respect of which they seek to enter information (i.e., drop a pin) in relation to a person or event.

A user may prefer to enter information relating to themselves onto the system database such as an autobiography, or some other person of interest, and make themselves contactable by providing their contact details, for example, a link to their Facebook page. In order to drop a pin and enter such information, the user navigates Google Maps by dragging their electronic input device (i.e., pointer) over the map in order to identify a geographic location of interest (latitude/longitude coordinates), for example, of their current residence, to which their profile is to be pinned.

Once the geographic location has been identified, the user may then “drop” a pin by selecting a location with their electronic input device designating the desired geographic location. Preferably, the information pin includes a URL link to their social media profile which is auto-populated upon the user “dropping” the pin. The user may also prefer to upload, or attach, a family tree, photographs and enter comments, facts, and/or anecdotes about the person of interest. Once the user saves the information, the information pin and associated information is available for identification by other users.

The user may continue to “drop” further pins or may elect to search for further information relating to the same person or other persons or events of interest, or may elect to exit the system.

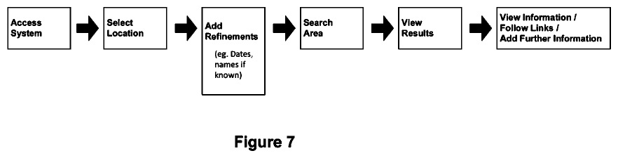

The steps involved in a user searching for information within a system in accordance with an embodiment of the invention is shown in FIG. 7.

The first step involves accessing the system through a user interface associated with the system that is available on the internet. The user is able to access the system by launching a browser and entering a URL upon which the user is presented with a login page as shown in FIG. 8.

If authentication of the user is required, the user will be prompted to enter their username and password. Upon providing the correct username and password details, the user is directed to the system profile which may contain personal details of the user such as their name and any links to associated social media profiles which could be used to auto-populate the information pins.

Once the user is directed to their system profile, the user will typically need to select a geographic location within which they propose to search for information.

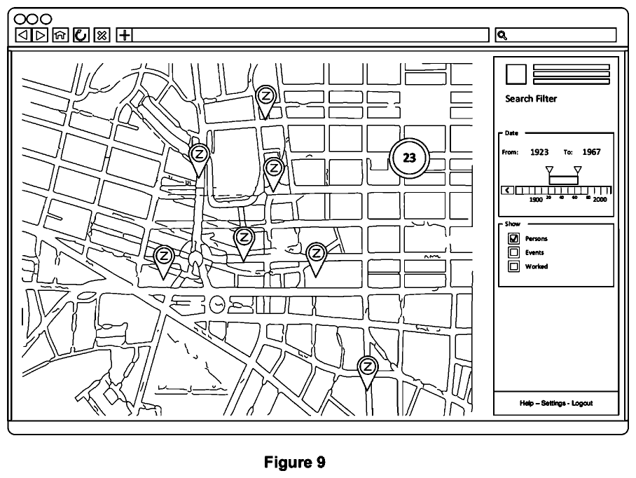

As a means of focusing or limiting the search results returned, a user may refine their search by entering search criteria such as a specific date range within a search filter located on the user interface. An example of a search filter according to an embodiment is shown in the interface in FIG. 9.

Once the desired geographic location has been selected and search criteria have been entered in the search filter, the user searches for information associated with persons of interest by navigating Google Maps and dragging their electronic input device (or pointer) over the map to locate a position of interest (latitude/longitude coordinates), for example, a known former residence of a deceased family member. In an embodiment, search criteria relating to the time period over which the search should occur is also selected using an electronic input device (or pointer) to define a time period.

As the user navigates the system, a search query is performed on the spatial database to identify any results/records in the search area that match the search (filtering) criteria. These records/results are overlaid on the map as shown in FIG. 9 which are visible to the user and which, when selected, provide information about the person of interest as is shown in the example of FIG. 5.

The user performing the search is able to enter their contact details and associate same with any saved record to facilitate communication with the user who initially pinned the information, or any other users having an interest in the same person. Alternatively, the user performing the search may elect to directly contact the user who initially pinned the information (preferably through a link connected to the social media profile), in an attempt to initiate a discussion and/or seek permission to update the information relating to the person of interest.

In the instance where there are numerous results to display in a geographic area, a summary marker is shown at that particular location (for example, the summary marker (23) shown in FIG. 9), that indicates how many records (pins) exist at the location that matches the entered search or filter criteria. The user may refine a search to further limit the results/records returned.

Accommodating the possibility of numerous elevations at a particular geographic location at the time of receiving information enables the system to graphically identify different elevations when a search result includes more than one relevant result at the same location. For example, the search results at different elevations for the same geographical location may be graphically represented in different colored pins or may be identified with a graphical representation of a stack of pins. The stack of pins may be unstacked by selection of the stack of pins with the user's electronic pointing device and selection of a specific unstacked pin to display information relating to a particular person or event is subsequently possible.

Once information relating to a person or event of interest has been identified, the user may continue to search for information to identify further information relating to persons, or events, of interest or may “drop” one or more pins in order to make themselves contactable or may elect to exit the system.

Throughout this specification and claims which follow, unless the context requires otherwise, the word “comprise”, and variations such as “comprises” and “comprising”, will be understood to imply the inclusion of a stated feature or step, or group of features or steps, but not the exclusion of any other feature or step or group of features or steps.

It will be appreciated by persons skilled in the relevant field of technology that numerous variations and/or modifications may be made to the invention as detailed in the embodiments without departing from the spirit or scope of the invention as broadly described. The present embodiments are therefore to be considered in all aspects as illustrative and not restrictive.

What is claimed is:

1. A computer implemented system enabling the discovery of personal contact details relating to a first user associated with a person or event of interest, the system including:
a user interface;
a geographical image; and
a mapping application;
wherein the user interface enables the first user to:
enter their personal contact details to the geographical image for discovery by one or more second users;
navigate the coordinate system by the use of the mapping application to identify a person or an event of interest, the first user able to identify the person or event of interest without requiring, prior to navigating the coordinate system, the name of the person or event; and
enter, amend and/or delete information in a database, relating to the identified person or event including a date or date range associated with the information; and
wherein the user interface enables the one or more second users to:
enter a second date or date range of interest into a search field and navigate the geographical image using the mapping application according to the second date or date range; and
thereby discover the entered personal details pertaining to the first user when the second date or date range matches the first date or date range.

2. A computer implemented system according to claim 1, wherein the information relating to the identified person includes a genealogical chart.

3. A computer implemented system according to claim 2, wherein the genealogical chart displays the contact details of one or more members within the genealogical chart.

4. A computer implemented system according to claim 1, wherein the information relating to the identified person includes a medical history.

5. A computer implemented system according to claim 4 wherein the medical history is a family medical history.

6. A computer implemented system according to claim 1, wherein the information relating to the identified person includes a cause of death.

7. A computer implemented system according to claim 7, wherein the personal contact details of the first user includes any one or more of a name, telephone number, email address, postal address, LinkedIn profile or Facebook page.

8. A computer implemented system according to claim 1, wherein the one or more second users are also able to enter, amend and/or delete information relating to the identified person or event in respect of which information has been entered by the first user.

9. A computer implemented system according to claim 1, wherein the information relating to the identified person or event includes a network of associations associated with the identified person or event.

10. A computer implemented system according to claim 9, wherein the network of associations includes the contact details of one or members within the network.

11. A computer implemented system according to claim 9, wherein the network of associations includes a genealogical chart.

12. A computer-implemented method for enabling the discovery of personal contact details relating to a first person associated with a person or event of interest, the method including:
the first user:
accessing, by a user interface configured to display a geographical image, a computer-implemented system;
navigating the coordinate system, by the use of a mapping application, to identify a person or an event of interest, the user able to identify the person or event of interest without requiring, prior to navigating the coordinate system, the name of the person or event; and
entering, amending and/or deleting information relating to the identified person or event in a database using the mapping application associated with the coordinate system; and
the one or more second users:
entering a second date or date range of interest into a search field and navigating the geographical image using the mapping application according to the second date or date range; and
thereby discovering the entered personal details pertaining to the first user when the second date or date range matches the first date or date range.

13. A computer implemented method according to claim 12, wherein the information relating to the identified person includes a genealogical chart.

14. A computer implemented method according to claim 13, wherein the genealogical chart displays the contact details of one or more members within the genealogical chart.

15. A computer implemented method according to claim 12, wherein the information relating to the identified person includes a medical history.

16. A computer implemented method according to claim 15 wherein the medical history is a family medical history.

17. A computer implemented method according to claim 12, wherein the information relating to the identified person includes a cause of death.

18. A computer implemented method according to claim 12, wherein the personal contact details of the first user includes any one or more of a name, telephone number, email address, postal address, LinkedIn profile or Facebook page.

19. A computer implemented method according to claim 12, wherein the one or more second users are also able to enter, amend and/or delete information relating to the identified person or event in respect of which information has been entered by the first user.

20. A computer implemented system according to claim 12, wherein the information relating to the identified person or event includes a network of associations associated with the identified person or event.

21. A computer implemented system according to claim 20, wherein the network of associations includes the contact details of one or members within the network.

22. A computer implemented system according to claim 21, wherein the network of associations includes a genealogical chart.

23. A computer implemented system enabling information relating to persons or events of interest to be entered, amended and/or deleted by one or more users, the system including:
a user interface;
a coordinate system; and
a mapping application;
wherein the user interface enables a user of the one or more users to:
navigate the coordinate system by the use of the mapping application to identify a person or an event of interest, the user able to identify the person or event of interest without requiring, prior to navigating the coordinate system, the name of the person or event; and enter, amend and/or delete information in a database, relating to the identified person or event.

24. A computer implemented system enabling the discovery of personal contact information pertaining to a first user, the system including:
a user interface for use by first and second users;
a geographical image;
a mapping application;
wherein the user interface enables a first user to anchor their personal contact information and a first date or date range to the geographical image for discovery by one or more second users; and
wherein the user interface enables the one or more second users to enter a second date or date range of interest into a search field to search the geographical image using the mapping application thereby discovering anchored personal contact information pertaining to the first user when the second date or date range matches the first date or date range.
